(19) United States
(12) Patent Application Publication
(10) Pub. No.: US 2007/0264252 A1
(43) Pub. Date: Nov. 15, 2007

(54) Modulators of Ms4a Gene Products

(76) Inventor: Sau Wei Li, West Sussex (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/575,595

(22) PCT Filed: Oct. 22, 2004
(86) PCT No.: PCT/EP04/11975

§ 371 Date: Mar. 15, 2007

(30) Foreign Application Priority Data
Oct. 24, 2003 (GB) 0324888.7

(51) Int. Cl. A61K 39/395; A61K 31/00; A61K 48/00; G01N 33/00

(52) U.S. Cl. 424/130.1; 436/86; 514/1; 514/44

(57) ABSTRACT

A method of identifying a substance suitable for use in the treatment of a leukocyte-associated inflammatory disease that modulates the activity of a polypeptide encoded by a human MS4A gene. The method involves combining a candidate substance with said polypeptide and measuring the effect of the candidate substance on the activity of said polypeptide. The polypeptide preferably forms a human ion channel, especially a CD20 ion channel.

The present invention relates to the identification of substances or agents that modulate the activity of membrane-spanning four-domains, sub-family A (MS4A) gene proteins, particularly CD20-and CD20-related proteins, and the use of such substances in the treatment of inflammatory diseases, particularly those of the respiratory system.

A variety of cells are attracted into tissues during inflammation. These include various leukocytes, particularly inflammatory phagocytes such as neutrophilic and eosinophilic granulocytes and monocytes. Mast cells, B-lymphocytes, CD4+ lymphocytes and eosinophils have been associated with respiratory diseases such as asthma and allergic rhinitis. Macrophages, CD8+ lymphocytes and neutrophils have been associated with inflammation and tissue destruction in respiratory diseases such as chronic obstructive pulmonary disease (COPD), including chronic bronchitis and emphysema associated therewith, and adult respiratory distress syndrome (ARDS), inflammatory bowel diseases such as Crohn's disease and ulcerative colitis, and rheumatoid arthritis.

Critical steps in the action of leukocytes in inflammatory conditions include the migration of these cells into the tissues, e.g. into the airways in respiratory inflammations or to the joints in rheumatoid arthritis, cell activation and the release of a range of inflammatory mediators, leukotrienes, oxygen radicals, proteases. Signals that are needed for leukocyte migration and activation are often communicated through receptors that respond to an increase in the level of cytosolic calcium.

Ins(1,4,5)P3 receptors are known to release calcium ions from intracellular stores but less is known about the channels in the plasma membrane through which those ions pass. Activation of the B cell receptor (BCR) in B lymphocytes leads to increases in calcium ion influx through plasma membrane calcium-permeable channels required for cytokine and IgE release. IgE binds to the high affinity IgE receptor of mast cells triggering mast cell activation characterised by an influx of extracellular calcium, which is essential for subsequent release of both preformed (granule-derived) mediators and newly-generated autacoids and cytokines (Church et al., 1982). Activation of T-lymphocytes via the T cell receptor (TCR) also elicits calcium influx which is essential for IL-2 generation and proliferation. The calcium entry pathways in mast cells and lymphocytes have been intensively studied electrophysiologically and shown to be mediated by a highly calcium-selective ion channel, the current identified in these studies is known as I_{CRAC}, calcium release-activated calcium current. The identity of CRAC channels in mast cells and lymphocytes is currently unknown however members of the family of 4 membrane-spanning proteins related to CD20 are molecular candidates for the channel subunits which form the CRAC channels responsible for the ICRAC currents in leukocytes.

Membrane-spanning four-domains, sub-family A is a gene superfamily designated MS4A, whose gene products include the B-cell-specific antigen CD20, hematopoietic-cell-specific protein HTm4 and high affinity IgE receptor beta chain FcεRIβ (Liang & Tedder, Genomics, vol 72, pp 119-127, 2001 and Ishibashi et al., Gene, vol 264, pp 87-93, 2001). These proteins share a similar structure with amino acid identities of 25-40%, the common structure being 4 transmembrane spanning domains with cytoplasmic N- and C-terminal domains. This topology is similar to many cation channel subunits, both pore-forming proteins such as the twin pore potassium channel proteins, and channel accessory subunits like the beta and gamma subunits of L-VOCC channels. A common feature of members of the MS4A family is a proline rich region in the N- and C-terminal domains which are reminiscent of the proline-rich regions in the cytoplasmic C-terminal domains of the TRPC and TRPM subfamilies of the transient receptor potential (TRP) cation channel gene family. These regions may mediate interactions with scaffolding proteins to form signalling complexes.

The best-functionally characterised members of the MS4A family are CD20 and FcεRIβ. CD20 is differentially expressed in B lymphocytes and FcεRIβ is particularly highly expressed in mast cells and basophils. Interestingly in terms of sequence homology, CD20 is the most divergent and also the largest member and has an unusually large number of residues between the third and fourth transmembrane domains (TMDs) compared with the other members of the family, this last feature may represent a putative pore region. MS4A6 and MS4A7 have the second longest chains of residues between the third and fourth TMDs. MS4A1, 6 and 7 are unusual within this family as the other members of the family have a short linking peptide chain between these regions.

The current carried by heterologously-expressed CD20 channels has an estimated reversal potential of >+60 mV suggesting that it is highly Ca²⁺ selective. The estimated single channel conductance in excised patches is 7 pS. The size of this current is much higher than that calculated for endogenous calcium release-activated calcium currents (I_{CRAC}) in T lymphocytes (˜10 fS for Ca²⁺) and mast cells, however the equivalent electrophysiological data for endogenous CD20 in B cells is not available and therefore the characteristics of CRAC currents may be different in B cells. It is likely that the channel composition of CRAC channels are multimeric and not identical in all cell types.

It is proposed in accordance with the present invention that MS4A members interact with each other, in homo- or hetero-multimeric complexes which may also include other protein subunits to form signalling complexes which regulate and/or mediate calcium influx. Furthermore as their distribution is largely leukocyte-restricted, MS4A proteins may be particularly important components of the Ca²⁺ entry pathways in leukocytes.

It is also proposed in accordance with the present invention that substances that attenuate the activation of leukocytes such as lymphocytes or mast cells by inhibiting the influx of calcium ions into such cells are useful for treating inflammatory diseases including respiratory diseases such as asthma and chronic obstructive pulmonary disease or may be used as immuno-suppressive agents e.g. to treat recipients of organ transplants to prevent tissue rejection. The present invention thus provides MS4A genes, particularly CD20 and closely-related genes, as therapeutic targets for such diseases and an assay for identifying MS4A modulators i.e. candidate compounds or agents including peptides, peptidomimetics, small molecules or other drugs, which stimulate or inhibit the activity of the pore-forming or accessory channel subunit proteins encoded by these genes, which may have therapeutic utility for inflammatory diseases or as immunosuppressive agents.

Accordingly, in a first aspect the present invention relates to a method of identifying a substance suitable for use in the treatment of a leukocyte-associated inflammatory disease which modulates the activity of a polypeptide encoded by a human MS4A gene, wherein the method comprises combining a candidate substance with said polypeptide and measuring the effect of the candidate substance on the activity of said polypeptide. The polypeptide preferably forms a human ion channel. It is preferably a product of the CD20 gene.

In a second aspect the present invention relates to a pharmaceutical composition comprising a compound that inhibits the influx of calcium ions through a human ion channel formed by a MS4A gene product and a pharmaceutically acceptable carrier. The MS4A gene product is preferably a CD20 gene product.

In a third aspect the present invention relates to the use of an antibody which is immunoreactive with a polypeptide encoded by a human MS4A gene, an antisense oligonucleotide comprising a nucleotide sequence complementary to a polynucleotide comprising a nucleotide sequence encoding that polypeptide, or a polynucleotide probe comprising at least 15 consecutive nucleotides of that polynucleotide, in the preparation of a pharmaceutical that inhibits the accumulation of leukocytes in human tissue.

In a fourth aspect the present invention relates to the use of an antibody which is immunoreactive with a polypeptide encoded by a human MS4A gene, an antisense oligonucleotide comprising a nucleotide sequence complementary to a polynucleotide comprising a nucleotide sequence encoding that polypeptide, or a polynucleotide probe comprising at least 15 consecutive nucleotides of that polynucleotide, in the preparation of a pharmaceutical for the treatment of a leukocyte-associated inflammatory disease.

In a fifth aspect the present invention relates to the use of a human MS4A inhibitor in the preparation of a pharmaceutical for the treatment of a leukocyte-associated inflammatory disease.

Throughout this specification and in the claims that follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

As mentioned above, in a first aspect the present invention relates to a method of the present invention for identifying a substance suitable for use in the treatment of a leukocyte-associated inflammatory disease which modulates the activity of a polypeptide encoded by a human membrane-spanning four-domains, sub-family 4 (herein “MS4A”) gene protein, e.g. CD20 or closely-related genes. In broad terms this method, or assay, comprises combining a candidate substance with a polypeptide encoded by a human MS4A gene and measuring the effect of the candidate substance on the activity of that polypeptide.

Substances that are suitable for use in the treatment of a leukocyte-associated inflammatory disease will tend to be enhancers (herein “human MS4A activators”) or inhibitors (herein “human MS4A inhibitors”) of human MS4A genes or human MS4A gene products.

The activity of a MS4A gene product may be measured, for example, by a cell-based method or screening assay that identifies compounds which have a stimulatory or inhibitory effect on the activity of human MS4A channels, e.g. CD20 channels, or by an appropriate reporter gene assay.

The abovementioned screening method may be carried out, for example, by preparing cells which express a MS4A polypeptide on their surfaces, e.g. insect, mammal or yeast cells and then incubating the resulting cells with the candidate substance to determine the enhancement or inhibition of a functional activity of a MS4A polypeptide.

In a suitable test for any activation of a MS4A channel, a candidate substance is combined with cells that are stably transfected with MS4A and express a functional MS4A polypeptide. Ion channel activity is measured by direct electrophysiological determination of MS4A-dependent cation currents or quantifying MS4A-mediated Ca²⁺ influx.

MS4A is activated by a variety of cells. These include cells that express G protein-coupled calcium mobilising receptors e.g. purinergic receptors, B cell receptors (BCR) or growth factor receptors e.g. insulin-like growth factor I (IGF-I), endogenous or transfected. They are therefore suitable to test for any inhibition of the MS4A protein. A receptor agonist e.g. ATP, an activator of BCR but also an antibody which directly binds to and activates MS4A can be used to activate MS4A-mediated cation channel activity or calcium influx. MS4A-mediated cation channel activity or calcium influx can also be monitored by measuring depletion of intracellular stores by treatment with a sarco-endoplasmic reticulum calcium-ATPase (SERCA) inhibitor e.g. thapsigargin or by reducing extracellular calcium concentrations. The cells are treated with the candidate substance either prior to or after stimulation of MS4A activity.

Electrophysiology is the gold standard functional assay for measurement of ion channel activity. It is performed, for example, using the methodology disclosed by Hamill, O. P. et al. (1981) in “Improved patch-clamp techniques for high-resolution current recording from cells and cell-free membrane patches” Pflugers Arch. (1981) 391, pages 85-100) or by CD20 electrophysiology methodologies disclosed by Bubien et al in “Transfection of the CD20 cellsurface molecule into ectopic cell types generates a Ca++ conductance found constitutively in B lymphocytes” Journal of Cell Biology (1993) Vol 121, no. 5, by Kanzaki et al in “Activation of a calcium-permeable cation channel CD20 expressed in Bab/c 3T3 cells by insulin-like growth factor-I” Journal of Biological Chemistry (1997) Vol 272, no. 8, or by Kanzaki et al in “Activation of the calcium-permeable cation channel CD20 by alpha subunits of the Gᵢ protein” Journal of Biological Chemistry (1997) Vol 272, no. 23.

Activation of MS4A leads to an increase in MS4A-mediated cation channel conductance and the magnitude of the current is reduced if the candidate substance inhibits the activity of MS4A. Alternatively, calcium influx is measured using fluorimetric-calcium dye based assay technologies for instance using Fluo-4 as the calcium-indicator. MS4A-mediated calcium influx should produce an increase in fluorescence, and the magnitude of the increase is reduced if the candidate substance inhibits the activity of MS4A. Any change in fluorescence is measured using suitable equipment, for example a fluorescence imaging plate reader.

Various known assay formats are suitable for screening activators and inhibitors of MS4A-dependent ion channel activity for instance in a high-throughput screening (HTS) for instance using fluorescence-based methods such as FLIPR, VIPR, ion flux assays and automated electrophysiology. Preferred assays are described by Xu et al. in “Ion-channel assay technologies: quo vadis?” (2001) Drug Discovery Today, Vol 6, 1278-1287, and by Wang et al in “Automated electrophysiology: high throughput of art” Assay. Drug Dev Technol (2003), vol 1, pages 695-708.

The present invention also relates to a pharmaceutical composition that comprises a compound that inhibits the influx of calcium ions through a MS4A channel and a pharmaceutically acceptable carrier.

One can use an antibody which is immunoreactive with the polypeptide encoded by a human MS4A gene (herein a “human MS4A antibody”) or an antisense oligonucleotide comprising a nucleotide sequence complementary to the polynucleotide comprising a nucleotide sequence encoding that polypeptide (herein a “human MS4A antisense oligonucleotide”), to prepare pharmaceuticals that inhibit the accumulation of leukocytes in human tissue.

The aforementioned human MS4A antibodies and antisense oligonucleotides may be used to treat leukocyte-associated inflammatory diseases.

Human MS4A activators, human MS4A inhibitors, human MS4A antibodies and human MS4A antisense oligonucleotides are hereinafter alternatively referred to collectively as “agents of the invention”.

Mast cell, basophil, B lymphocyte, T lymphocyte and eosinophil-associated inflammatory diseases to which the present invention is applicable include inflammatory or obstructive airways diseases, particularly asthma and allergic rhinitis, and also other allergic diseases including dermatological allergic diseases. Other lymphocyte-associated inflammatory diseases to which the present invention is also applicable include lymphocyte-associated inflammatory or obstructive airways diseases, particularly chronic obstructive pulmonary disease (COPD), including chronic bronchitis and emphysema, and acute (or adult) respiratory distress syndrome (ARDS). Further lymphocyte-associated inflammatory diseases to which the present invention is also applicable include rheumatoid arthritis and inflammatory bowel diseases such as Crohn's disease and ulcerative colitis.

CD20 is expressed in B-cell precursors and mature B cells. HTm4 is expressed in diverse lymphoid- and myeloid-origin haematopoietic cells including mast cells. FcεRIβ is expressed in mast cells and basophils. It is proposed in accordance with the present invention that substances that attenuate the activation of leukocytes such as mast cells, B lymphocytes and T lymphocytes by inhibiting the influx of calcium ions into such cells are useful for treating respiratory diseases such as asthma and chronic obstructive pulmonary disease. These substances may also be used as immunosuppressive agents e.g. to treat recipients of organ transplants to prevent tissue rejection or to treat autoimmune diseases e.g. multiple sclerosis.

A human MS4A polypeptide, such as a CD20 protein or closely-related protein, can be isolated using any suitable conventional method. Since the sequences for certain human MS4A genes are known, e.g. the human CD20 gene, specific primers may be used as a convenient option.

A human MS4A polynucleotide may be cDNA, genomic DNA or RNA and may be obtained using any suitable conventional method. For example it may be prepared from the nucleotides which it comprises by chemical synthesis, e.g. automated solid phase synthesis using known procedures and apparatus.

A human MS4A antibody may be a polyclonal or monoclonal antibody. Such antibodies may be prepared using conventional procedures. Methods for the production of polyclonal antibodies against purified antigen are well established (cf. Cooper and Paterson in Current Protocols in Molecular Biology, Ausubel et al. Eds., John Wiley and Sons Inc., Chapter 11). Human MS4A antibodies may be used to detect, or determine the level of expression of, human MS4A polypeptides, or to inhibit ligand/anti-ligand binding activities of human MS4A polypeptides.

A human MS4A antisense oligonucleotide may be DNA, an analogue of DNA such as a phosphorothioate or methylphosphonate analogue of DNA, RNA, an analogue of RNA, or a peptide nucleic acid (PNA). The antisense oligonucleotide may be synthesised by conventional methods, for example using automated solid phase techniques. It may be used to inhibit the expression of a human MS4A gene, e.g. the CD20 gene, where this is desired. Alternatively, a short interfering RNA (siRNA) can be used as a specific tool for targeted gene knockdown. RNA interference inhibits gene expression and can therefore be used to explore gene function. This technique is described by S. M. Elbashir et al in Methods 26 (2002) 199-213.

A human MS4A polynucleotide probe comprises at least 15 contiguous nucleotides of the aforementioned polynucleotide or a complement thereof. The probe may be cDNA, genomic DNA or RNA and can be synthesised by conventional methods. Usually it is a synthetic oligonucleotide comprising 15 to 50 nucleotides, which can be labelled, e.g. with a fluorophore, to provide a detectable signal. A human MS4A polynucleotide probe can be used to detect the presence or absence of a human MS4A gene, e.g. the human CD20 gene, i.e. to detect genetic abnormality.

The effectiveness of an agent of the invention in inhibiting inflammatory conditions, for example in inflammatory airways diseases, may be demonstrated in an animal model, e.g. a mouse or rat model, of airways inflammation or other inflammatory conditions, for example as described by Szarka et al, J. Immunol. Methods (1997) 202:49-57; Renzi et al, Am. Rev. Respir. Dis. (1993) 148:932-939; Tsuyuki et al., J. Clin. Invest. (1995) 96:2924-2931; and Cernadas et al (1999) Am. J. Respir. Cell Mol. Biol. 20:1-8.

The effectiveness of an agent of the invention in inhibiting or reversing a leukocyte-associated inflammatory disease may be demonstrated in a model of the disease, e.g. a lipopolysaccharide-induced lung inflammation model in rat or mouse or models described by Durie et al., Clin. Immunol. Immunopathol. (1994) 73: 11-18; and Williams et al, Proc. Natl. Acad. Sci. USA (1992) 89: 9784-9788.

The agents of the invention may be administered by any appropriate route, e.g. orally, for example in the form of a tablet or capsule; parenterally, for example intravenously; topically, e.g. in an ointment or cream; transdermally, e.g. in a patch; by inhalation; or intranasally.

Pharmaceutical compositions containing agents of the invention may be prepared using conventional diluents or excipients and techniques known in the galenic art. Thus oral dosage forms may include tablets and capsules, and compositions for inhalation may comprise aerosol or other atomizable formulations or dry powder formulations.

When the composition comprises an aerosol formulation, it preferably contains, for example, a hydrofluoroalkane (HFA) propellant such as HFA134a or HFA227 or a mixture of these, and may contain one or more co-solvents known in the art such as ethanol (up to 20% by weight), and/or one or more surfactants such as oleic acid or sorbitan trioleate, and/or one or more bulking agents such as lactose. When the composition comprises a dry powder formulation, it preferably contains, for example, the compound of formula I having a particle diameter up to 10 microns, optionally together with a diluent or carrier, such as lactose, of the desired particle size distribution and a compound that helps to protect against product performance deterioration due to moisture e.g. magnesium stearate. When the composition comprises a nebulised formulation, it preferably contains, for example, the compound of formula I either dissolved, or suspended, in a vehicle containing water, a co-solvent such as ethanol or propylene glycol and a stabiliser, which may be a surfactant.

The invention includes (i) an agent of the invention in inhalable form, e.g. in an aerosol or other atomizable composition or in inhalable particulate, e.g. micronised form, (ii) an inhalable medicament comprising an agent of the invention in inhalable form; (iii) a pharmaceutical product comprising such an agent of the invention in inhalable form in association with an inhalation device; and (iv) an inhalation device containing an agent of the invention in inhalable form.

Dosages of agents of the invention employed in practising the present invention may of course vary depending, for example, on the particular condition to be treated, the effect desired and the mode of administration. In general, suitable daily dosages for administration by inhalation are of the order of 1 μg to 10 mg/kg while for oral administration suitable daily doses are of the order of 0.1 mg to 1000 mg/kg.

The contents of the articles cited in this patent specification are incorporated herein by reference.

1. A method of identifying a substance suitable for use in the treatment of a leukocyte-associated inflammatory disease which modulates the activity of a polypeptide encoded by a human MS4A gene, wherein the method comprises combining a candidate substance with said polypeptide and measuring the effect of the candidate substance on the activity of said polypeptide.

2. A method according to claim 1 wherein the polypeptide forms a human ion channel.

3. A method according to claim 2 wherein the polypeptide is a product of the CD20 gene.

4. A pharmaceutical composition comprising a compound that inhibits the influx of calcium ions through a human ion channel formed by a MS4A gene product and a pharmaceutically acceptable carrier.

5. A composition according to claim 4 wherein the MS4A gene product is a CD20 gene product.

6. (canceled)

7. (canceled)

8. A method according to claim 1, in which the disease is a mast cell, basophil, B lymphocyte, lymphocyte or eosinophil-associated disease.

9. A method according to claim 8, in which the mast cell, basophil, B lymphocyte, lymphocyte or eosinophil-associated disease is asthma or allergic rhinitis.

10. A method according to claim 1, in which the disease is a macrophage, lymphocyte, neutrophil-associated disease such as chronic obstructive pulmonary disease, adult respiratory distress syndrome, rheumatoid arthritis or inflammatory bowel disease.

11. A method of treating a leukocyte-associated inflammatory disease in a subject in need of such treatment, which comprises administering to said subject an effective amount of a human MS4A inhibitor or a human CD20 protein inhibitor.

12. A method of treating a condition mediated by inhibiting the accumulation of leukocytes in human tissue in a subject in need of such treatment, which comprises administering to said subject an effective amount of an antibody which is immunoreactive with a polypeptide encoded by a human MS4A gene, an antisense oligonucleotide comprising a nucleotide sequence complementary to a polynucleotide comprising a nucleotide sequence encoding that polypeptide, or a polynucleotide probe comprising at least 15 consecutive nucleotides of that polynucleotide.

13. A method of treating a leukocyte-associated inflammatory disease in a subject in need of such treatment, which comprises administering to said subject an effective amount of an antibody which is immunoreactive with a polypeptide encoded by a human MS4A gene, an antisense oligonucleotide comprising a nucleotide sequence complementary to a polynucleotide comprising a nucleotide sequence encoding that polypeptide, or a polynucleotide probe comprising at least 15 consecutive nucleotides of that polynucleotide.
